(19) United States
(12) Patent Application Publication
(10) Pub. No.: US 2002/0052420 A1
(43) Pub. Date: May 2, 2002

(54) Method and composition for modulating an immune response

(76) Inventors: George Hasko, Salem, MA (US); Csaba Szabo, Gloucester, MA (US)

Correspondence Address: MINTZ, LEVIN, COHN, FERRIS,, GLOVSKY and POPEO, P.C., One Financial Center, Boston, MA, 02111, US

(21) Appl. No.: 09/986,206

(22) Filed: Oct. 19, 2001

Related U.S. Application Data
(62) Division of application No. 09/626,602, filed on Jul. 27, 2000.
(63) Continuation-in-part of application No. 09/491,888, filed on Jan. 24, 2000.
(63) Continuation-in-part of application No. 09/452,427, filed on Dec. 1, 1999.
(60) Provisional application No. 60/110,562, filed on Dec. 2, 1998.

(51) Int. Cl. A61K 31/045

(52) U.S. Cl. 514/729

(57) ABSTRACT

Disclosed is a method of inhibiting or preventing a condition associated with undesired secretion of a macrophage inflammatory protein using inhibitors of ATP-sensitive K⁺-channels, inhibitors of the Na⁺/H⁺ antiporter, inosine, or inosine analogs.

### RELATED APPLICATIONS

This application claims priority to U.S. Ser. No 09/491,888, filed Jan. 24, 2000; U.S. Ser. No. 09/452,427, filed Dec. 1, 1999; U.S. Ser. No. 60/110,562, filed Dec. 2, 1998; and to U.S. Ser. No. 09/626,602, filed Jul. 27, 2000. The contents of these applications are incorporated herein by reference in their entirety.

### FIELD OF THE INVENTION

The invention relates to a method and composition for treatment of a condition associated with undesired secretion of a macrophage inflammatory protein.

### BACKGROUND OF THE INVENTION

Various forms of inflammation are characterized by activation of macrophages. Macrophages are thought to induce and maintain inflammatory processes mainly by producing various products which, by acting on other cells, bring about the deleterious consequences of inflammation. For example, macrophages produce cytokines. These proteins are central mediators in inflammatory processes, such as the local inflammatory processes characteristic of arthritis or colitis. Cytokines produced by macrophages are also thought to be involved in systemic inflammatory processes, such as endotoxic shock. Macrophage products are more generally involved in pathophysiological mechanisms, such as plasma extravasation, inflammatory cell diapedesis, release of toxic free radicals, endothelial injury, and release of tissue degrading enzymes, which can result in tissue injury and, ultimately, organ failure.

Tumor necrosis factor (TNF) is another cytokine associated with macrophage activation TNF is also thought to be involved in inducing most of the pathophysiological events characteristic of inflammation. For example, TNF is a key cytokine in the toxic effect of endotoxin (LPS) and in the pathogenesis of septic shock, as evidenced by high serum plasma levels of TNF after LPS administration to animals or to human volunteers, or in septic subjects. Administration of anti-TNF antibodies protects against the lethal effects of LPS and of live bacteria in a variety of animal models. Moreover, TNF can be a central target in the treatment of rheumatoid arthritis

Interleukin-12 (IL-12) is another macrophage product which has been shown to be involved in the induction of pathology in several inflammatory diseases. These diseases include autoimmune diseases such as multiple sclerosis, inflammatory bowel disease, insulin dependent diabetes mellitus, and rheumatoid arthritis; and inflammatory states such as septic shock and the generalized Schwarzman reaction. For example, administration of anti-IL-12 antibodies substantially reduces the incidence and severity of adoptively transferred experimental allergic encephalomyelitis, suggesting that endogenous IL-12 is involved in its pathogenesis. Furthermore, the course of disease in adjuvant-induced arthritis is suppressed in IL-12 deficient mice, or in mice treated with anti-mIL- 12 antibodies.

The chemokine macrophage inflammatory protein (MIP)-1α and the CXC chemokine MIP-2 are additional proinflammatory proteins expressed by macrophages.

### SUMMARY OF THE INVENTION

The invention is based in part on the discovery that various small molecules inhibit the release of macrophage inflammatory proteins. Accordingly, the invention provides a method of treating a subject having or at risk for a condition associated with undesired secretion of a macrophage inflammatory protein. The method includes administering an immunomodulator that is an inhibitor of K_{ATP} channels, an inhibitor of a Na+/H+ transporter, inosine, or an analog thereof.

In one aspect, the invention includes a method of treating a subject having, or at risk for, a condition associated with undesired secretion of a macrophage inflammatory protein (MIP). The method includes administering to the subject an immunomodulator in an amount sufficient to treat or delay the onset of the condition. The immunomodulator can be, for example, a K_{ATP} channel inhibitor, an inhibitor of a Na⁺/H⁺ exchanger, inosine, or an inosine analog.

The condition associated with undesired secretion of a MIP can be, e.g., inflammation, shock, or both. The inflammation can be associated with a condition such as e.g., diabetes mellitus (including autoimmune diabetes), adult respiratory distress syndrome, arthritis, vasiculitis, autoimmune disease, lupus erythematosus, ileitis, ulcerative colitis, Crohn's disease, asthma, gingivitis, periodontitis, ophthalmitis, endophthalmitis, nephrosis, AIDS-related neurodegeneration, stroke, neurotrauma, Alzheimer's disease, encephalomyelitis, cardio-myopathy, transplant rejection, and cancer.

Examples of conditions associated with shock include shock caused by, or associated with, gram positive bacteria-mediated circulatory shock, gram negative bacteria-mediated circulatory shock, hemorrhagic shock, anaphylactic shock, systemic inflammation, pro-inflammatory cytokines, and systemic inflammatory response syndrome (SIRS).

The immunomodulator can be administered via, e.g., intravenous, intramuscular, subcutaneous, sublingual, oral, rectal or aerosol delivery. Administration of the immunomodulator can be prophylactic, therapeutic, or both.

The immunomodulator can be e.g., a K_{ATP} channel-blocking inhibitor. In some embodiments, the inhibitor inhibits a macrophage K_{ATP} channel. An example of a K_{ATP} channel inhibitor is a sulphonylurea compound, such as glibenclamide.

In some embodiments, the immunomodulator is an inhibitor of a Na⁺/H⁺ exchanger, e.g., a pyrazinoylguanidine derivative or a pteridine derivative. An example of a pyrazinoylguanidine derivative immunomodulator is amiloride. An example of a pteridine derivative immunomodulator is triamterene.

In some embodiments, the immunomodulator is one or more of amiloride, inosine, an inosine analog, glibenclamide, 5-(N,N-dimethyl)-amiloride hydrochloride; 5-(N-ethyl, N-isopropyl)-amiloride; 5(N, N-hexamethylene)-amiloride; 5-(N-methyl, N-isobutyl)-amiloride; benzamil, tolbutamide; glipizide; 2,3-butanedione monoxime; meglitinide.

The subject can be, e.g., a mammal, such as a rat, mouse, rabbit, guinea pig, hamster, cow, pig, horse, goat, sheep, dog, cat, non-human primate, or human.

In a further aspect, the invention includes a method for treating or preventing diabetes, e.g., autoimmune diabetes, by administering to a patient in need of such treatment a safe and therapeutically effective amount of inosine, or an inosine receptor ligand, e.g., a compound which binds to an inosine binding site.

The immunomodulator can be administered via, e.g., intravenous, intramuscular, subcutaneous, sublingual, oral, rectal or aerosol delivery. Administration of the immunomodulator can be prophylactic, therapeutic, or both.

The subject can be, e.g., a mammal, such as a rat, mouse, rabbit, guinea pig, hamster, cow, pig, horse, goat, sheep, dog, cat, non-human primate, or human.

Also provided is a method of increasing insulin levels in a subject. The method includes administering to a subject in need thereof an amount of inosine or a ligand for an inosine binding site in an amount sufficient to increase insulin levels in said subject. In preferred embodiments, administering the inosine or inosine receptor ligand to the subject increases pancreatic insulin levels in the subject.

The methods and pharmaceutical compositions described herein can used to inhibit or prevent secretion of inflammatory proteins such as TNF, IL-12, MIP-1α, and MIP-2. Because of the pivotal role of these proteins in the initiation and maintenance of inflammatory diseases, these cytokines are ideal targets for anti-inflammatory therapy in such disease states. The methods described herein can simultaneously inhibit release of multiple inflammatory proteins. Thus, because these inflammatory proteins act in distinct ways, higher therapeutic effectiveness can be obtained with the herein-described methods and compositions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic drawing showing the release of various cytokines over time following administration of inosine in mice.

FIG. 2 is a graph showing the number of mice surviving (y-axis) over time (x-axis) following exposure to challenge with LPS following pretreatment with drug vehicle (physiologic saline) or 100-mg/kg inosine.

### DETAILED DESCRIPTION OF THE INVENTION

The invention provides compositions and methods for treating disorders associated with undesired secretion of macrophage inflammatory proteins. The invention is based in part on the observations that: (a) inhibitors of the ATP-gated K+ channel inhibit secretion of inflammatory cytokines; (b) inhibitors of the Na+/H+ transporter inhibit secretion of inflammatory cytokines; and (c) the nucleoside inosine inhibits secretion of inflammatory cytokines.

Accordingly, in one aspect, the invention provides a method of treating a subject having or at risk for a condition associated with undesired secretion of a macrophage inflammatory protein. By “at risk for” is meant a state that negatively impacts a subject such that it has an increased likelihood of developing a condition associated with undesired secretion of a macrophage inflammatory protein. “Undesired” as used herein is secretion of an inflammatory protein that causes, or is otherwise associated with, an undesired physiological reaction in the subject. Inflammatory proteins include proteins such as TNF, IL-12, MIP-1α, MIP-2, or IFN-γ.

The method includes administering to the subject an immunomodulator in an amount sufficient to treat, or delay the onset of, the condition. The immunomodulator preferably inhibits secretion of two or more macrophage inflammatory proteins. Alternatively, or in addition, the immunomodulator inhibits secretion of one or more macrophage inflammatory proteins while promoting expression of one or more anti-inflammatory proteins. An example of a macrophage anti-inflammatory protein is IL-10.

In another aspect, the invention provides pharmaceutical compositions comprising one or more of the herein-described immunomodulators. The compositions can be used for treating a subject having or at risk for a condition associated with undesired secretion of the macrophage inflammatory protein. As used herein, an “immunomodulator” is a compound that modulates an immune response by inhibiting expression or activity of one or more macrophage inflammatory proteins. Expression can be inhibited, for example, by inhibiting secretion of the inflammatory proteins. Examples of immunomodulators include a K_{ATP} channel-blocking inhibitor, an inhibitor of a Na⁺/H⁺ exchanger, and/or inosine, or analogs thereof, including inosine receptor ligands. Preferably, the K_{ATP} channel-blocking inhibitor inhibits a macrophage K_{ATP} channel.

An example of a K_{ATP} channel-blocking inhibitor is a sulphonylurea compound. Examples of sulphonylurea compounds include glibenclamide, glipizide, and tolbutamide. This class of drugs, which is used to treat non-insulin dependent diabetes mellitus, is activated by the antihypertensive agents diazoxide or minoxidil. Accordingly, in some embodiments, the K_{ATP} channel-blocking inhibitors are administered with one or more of these antihypertensive agents.

Na+/H+ exchanger inhibitors can e.g., a pyrazinoylguanidine derivative or a pteridine derivative, or analogs thereof. Preferred pyrazinoylguanidine derivatives include amilioride or an amilioride analog, e.g., 5-(N, N-dimethyl)-amiloride hydrochloride; 5-(N-ethyl, N-isopropyl)-amiloride; 5(N, N-hexamethylene)-amiloride; 5-(N-methyl, N-isobutyl)-amiloride, benzamil, and 3,4-dichlorobenzamil. Preferred pteridine derivatives include, e.g., triamterene.

Preferred K_{ATP} channel inhibitors include, e.g. 2,3-butanedione monoxime; meglitinide, glipizide, tolbutamide, chlorprpopamide, tolazamide, gliclazide, and repaglinide.

Examples of inosine analogs include, e.g., 8-bromo-inosine, and 8-chloroinosine. Inosine analogs include those which bind to an inosine binding site, or are inosine receptor ligands.

The immunomodulators can be provided in a pure form or in a pharmaceutically acceptable carrier and can be used to treat or prevent conditions and disorders associated with undesired secretion of one or more macrophage inflammatory proteins.

For example, the immunomodulators may be used to treat, or to delay the appearance of, diseases associated with inflammation. Examples of such diseases include chronic inflammatory disorders of the joints including arthritis, e.g., rheumatoid arthritis and osteoarthritis, inflammatory bowel diseases such as ileitis, ulcerative colitis and Crohn's disease; inflammatory lung disorders such as asthma and chronic obstructive airway disease. Other examples of disorders include inflammatory disorders of the eye such as corneal dystrophy, trachoma, onchocerciasis, uveitis, sympathetic ophthalmitis and endophthalmitis. Disorders may also include chronic inflammatory disorders of the gum, e.g., periodontitis; tuberculosis, leprosy, inflammatory diseases of the kidney including glomerulonephritis and nephrosis; inflammatory disorders of the skin including sclerodermatitis, psoriasis and eczema; inflammatory diseases of the central nervous system, including AIDS-related neurodegeneration, stroke, neurotraua and Alzheimer's disease, encephalomyelitis and viral or autoimmune encephalitis; autoimmune diseases including immune-complex vasculitis, systemic lupus and erythematodes; systemic lupus erythematosus (SLE); and inflammatory diseases of the heart such as cardiomyopathy. Additional examples include adult respiratory distress syndrome, gingivitis, transplant rejection, and cancer.

Alternatively, or in addition, the condition can be shock. Shock in the subject may be associated with an underlying condition such as septic shock, e.g., gram positive bacteria-mediated circulatory shock, gram negative bacteria-mediated circulatory shock, hemorrhagic shock, anaphylactic shock, systemic inflammation, pro-inflammatory cytokines, and systemic inflammatory response syndrome (SIRS). The immunomodulators may also be used to prevent or treat circulatory shock, such as shock occurring as a result of gram-negative and gram positive sepsis, trauma, hemorrhage, burn injury, anaphylaxis, cytokine immunotherapy, liver failure, kidney failure or systemic inflammatory response syndrome.

In some embodiments, the immunomodulator is used to treat or prevent diabetes mellitus in a subject. The diabetic condition can be, e.g., Type I or Type II diabetes. The diabetic condition treated can be autoimmune diabetes. Autoimmune diabetes is associated with a strong inflammatory component, activation of macrophages and infiltration of mononuclear cells into the pancreas. The subsequent inflammatory processes bring about the deleterious consequences of inflammation diabetes, such as islet inflammation, islet cell destruction, insulin deficiency and hyperglycemia. Rabinovitch et al., Biochem. Pharmacol. 55:1139-49, 1998; Almawi et al., J Clin. Endocrinol. Metab. 84:1497-502, 1999. Macrophage-produced cytokines can be important mediators in the intraislet inflammatory processes. Accordingly, the herein-disclosed immunomodulators can be used to treat or prevent the development of a diabetic condition in a subject.

In another aspect, the invention provides a method for treating or preventing conditions or diseases associated with inflammatory bowel disease in a subject. The method includes administering a therapeutically effective amount of an immunomodulator, e.g., inosine or a ligand for an insosine binding site. The subject can be, e.g., a human.

The immunomodulator therapeutically or prophylactically and can be administered in any route recognized in the art. For example, administration can be intravenous, intramuscular, subcutaneous, sublingual, oral, rectal or by aerosol delivery. In some embodiments, the immunomodulator is administered to the subject in the form of a depot. Preferably, the depot increases the biological half-life of the immunomodulator.

Administration can be at a dose from about 0.1 to about 500 mg/kg/day of the immunomodulator in the subject. In various embodiments, the dose is, e.g., between about 0.5 to 250 mg/kg/day, 1.0 to 125 mg/kg/day, 5 to 75 mg/kg/day, 10 to 50 mg/kg/day, or 20 to 40 mg/kg/day.

If desired, the immunomodulator can be administered along with a second agent that itself is useful for treating conditions associated with inflammatory bowel disease. For example, the second agent can be an antibiotic, a glucocorticoid, an immunosuppressive agent, an aminosalicylate and a non-steroidal anti-inflammatory agent. Examples of second agents include, e.g., dexamethasone, 5-aminosalicylic acid, sulfasalazine, 4-aminosalicylic acid. sulphapyridine, 6-mercaptopurine, azathioprine, cyclosporine, anti-tumor necrosis factor antibody, soluble tumor necrosis factor receptor, and an anti-C5 antibody. If desired, the immunomodulator can be administered along with two or more, e.g., three, four, or five of the second agents.

Examples of inflammatory bowel diseases or conditions that can be treated according to the invention include, e.g., ileitis, ulcerative colitis, and Crohn's disease.

Also provided by the invention is a method of increasing inosine levels in a subject who has or is at risk of developing an inflammatory bowel disease. The method includes administering an amount of an immunomodulator (e.g., inosine or an analog of an inosine binding site) sufficient to increase inosine levels in the subject.

In another aspect, the invention includes a method of treating a subject suffering from or at risk for inflammatory bowel disease. The method includes administering to the bowel of the subject, e.g. in an enemator, a composition that includes a therapeutic amount of an immunomodulator and a bowel-compatible pharmacologically acceptable carrier. The composition is administered topically to the location of the inflammatory bowel disease in the bowel of the subject suffering from or at risk of inflammatory bowel disease.

In another aspect, the invention includes a pharmaceutical composition comprising a safe and therapeutically effective amount of inosine (or a ligand for an inosine binding site) and a pharmaceutically effective carrier. Preferably, the pharmaceutical composition is formulated for treating inflammatory bowel disease in a subject suffering from or at risk for inflammatory bowel disease. For example, the pharmaceutical composition can include one or more of a pharmacologically- and bowel-compatible carrier, adapted for delivery of the inosine (or ligand for an insosine binding site) to the bowel of the subject. Any carrier recognized in the art can be used. Examples of carriers include,(i) a foam suitable for rectal administration; (ii) a suppository base which surrounds the inosine (or ligand for an insosine binding site); and (iii) an orally ingestible time-release substance which withstands degradation by the gastric acids of the stomach and releases the inosine (or ligand for an inosine binding site) in the bowel.

The inosine or ligand for an inosine binding site can be present in the composition in a concentration ranging from, e.g., 0.2 to 15 grams, 1 to 20 grams, or 2 to 5 grams.

The foam in the pharmaceutical composition preferably includes inosine or a ligand for an inosine binding site, a surfactant, an adjuvant and a blowing agent. For example, the foam can include 0.5 to 5 grams of inosine as the active ingredient and 20 g of a foam containing propylene glycol, emulsifying wax, polyoxyethylene-10-stearyl ether, cetyl alcohol, methylparaben and propylparaben, trolamine, purified water and inert propellants, dichlorodifluoromethane or dichlorotetrafluoroethane.

The carrier in the pharmaceutical composition preferably includes one or more of propylene glycol, emulsifying wax, polyoxyethylene-10-stearyl ether, ethoxylated cetyl and stearyl alcohols, stearath-10, cetyl alcohol, methyl paraben, propyl paraben, trolamine, purified water, cetyl alcohol, ethoxylated stearyl alcohol, dry ethanolamine, de-ionized water and suitable propellents.

The suppository base in the composition preferably includes one or more of theobroma oil, glycerinated gelatin, hydrogenated vegetable oil, polyalkyl glycol, fatty acid ester of polyalkylene glycol, coconut oil base, hydrogenated fatty acid, monoglyceride, cocoa butter, petroleum oil, beeswax, glycerine, polyethylene glycol 600 dilaurate, hydrogenated cocoa glyceride and polyethylene glycol.

The timed release substance in the pharmaceutical composition can include one or more of an acrylic-based resin coating, a methacrylic acid copolymer, an acrylic-based resin mixed with a suitable non-medicinal carrier selected from the group consisting of lactose, magnesium stearate, polyethylene glycol, polyvinyl pyrolidone, or sodium starch glycolate, cellulose or ethyl cellulose, a matrix composition comprised of a hydrophilic polymer and an enteric polymer, a cellulose derivative, polyvinyl acetate phthalate, or polyvinyl acetate phthalate mixed with a plasticizer, a polysaccharide which is decomposable in the bowel, a locust bean gum or a guar gum, a film-forming polymer having hydrophilic groups, a film-forming acrylic polymer in admixture with a polysaccharide comprising from 30 to 100% by weight of at least one monomer selected from the group consisting of lower alkyl esters of acrylic acid and lower alkyl esters of methacrylic acid, a hydrocolloid gum obtained from a higher plant, and an anionic carboxylic polymer which does not dissolve at a pH below about 4 but is soluble at a pH ranging from about 4 to about 7.5.

The pharmaceutical composition can be provided as a coated polymer. For example, in one embodiment, the composition includes between about 0.1% by weight to about 90% by weight of an immunomodulator coated with about 5% by weight to about 29% by weight of a hydrophilic polymer, and from about 0.5% by weight to about 25% by weight of an acrylic polymer which dissolves at a pH in the range of about 5.0 to about 7.5.

In some embodiments, the pharmaceutical composition including the immunomodulator is present as a capsule or a tablet.

In some embodiments, the immunomodulator is enterically coated so as to be released in the terminal portion of the ileum and in the colon.

In some embodiments, the immunomodulator is present in a unit dosage form adaptable for oral administration. Preferably, the unit dosage form is effective to relieve a symptom of inflammatory bowel disease without dose-limiting systemic toxicity.

In various embodiments, the pharmaceutical composition is used to treat or prevent inflammatory bowel diseases such as, e.g., ulcerative colitis Crohn's disease, or ulcerative colitis.

Also provided by the invention is an enema formulation for treating or preventing a condition associated with inflammatory bowel disease (e.g., ulcerative colitis Crohn's disease, or ulcerative colitis). The formulation includes an amount of inosine or an inosine analog in an amount effective to relieve a symptom of inflammatory bowel disease without dose-limiting systemic toxicity.

In some embodiments, the formulation is provided in combination with a flowable carrier, which amount is released in the lower intestinal tract. The flowable carrier can be, e.g., water, alcohol, or an aqueous alcohol fluid. If desired, the flowable carrier can be thickened with one or more of gums, acrylates or modified celluloses.

The formulation may additionally include a lubricant or a foaming agent. The formulation in some embodiments if provided in a form suitable for delivery from a prefilled bag or syringe. If desired, the enema formulation can be provided in a form suitable for delivery from a pressurized container.

Preferably, the amount of inosine or ligand for an inosine binding site, in the formulation is about 150-1000 mg, e.g., about 200 to about 800 mg, about 300 to about 700 mg, or about 400 to about 650 mg.

The immunomodulator can be administered to the subject prophylactically or therapeutically. The subject can be, e.g., a mammal, such as a rat, mouse, rabbit, guinea pig, hamster, cow, pig, horse, goat, sheep, dog, cat, non-human primate, or human. The subject can have, or be at risk for developing, a condition associated with undesired secretion of one or more inflammatory cytokines.

The immunomodulator can be administered to the subject by any route that elicits the desired response, while preferably minimizing any undesirable side effects. Suitable routes can include intravenous, intramuscular, subcutaneous, sublingual, oral, rectal or aerosol delivery.

The macrophage immunomodulators are administered to a subject in need of treatment for the conditions described above in an effective amount. As used herein, “effective amount” defines that amount of pharmaceutical active which provides the desired therapeutic effect while providing an acceptable level of side effects (if any) for the subject.

In another aspect the invention includes pharmaceutical, or therapeutic, compositions containing one or more immunomodulators described herein. Pharmaceutical formulations may include those suitable for oral, rectal, nasal, topical (including buccal and sub-lingual), vaginal or parenteral (including intramuscular, sub-cutaneous and intravenous) administration, or for administration by inhalation or insufflation. The formulations may, where appropriate, be conveniently presented in discrete dosage units and may be prepared by any of the methods well known in the art of pharmacy. All such pharmacy methods include the steps of bringing into association the active compound with liquid carriers or finely divided solid carriers or both as needed and then, if necessary, shaping the product into the desired formulation.

Pharmaceutical formulations suitable for oral administration may conveniently be presented: as discrete units, such as capsules, cachets or tablets, each containing a predetermined amount of the active ingredient; as a powder or granules; or as a solution, a suspension or as an emulsion. The active ingredient may also be presented as a bolus electuary or paste, and be in a pure form, i.e., without a carrier. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration may be presented as a suppository with the usual carriers such as cocoa butter or polyethylene glycol. Formulations for topical administration in the mouth, for example buccally or sublingually, include lozenges, comprising the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents. Liquid sprays are conveniently delivered from pressurized packs.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insuffiator.

When desired, the above-described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the macrophage immunomodulator ligands may be administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The pharmaceutical composition preferably is administered orally or by injection (intravenous or subcutaneous), and the precise amount administered to a subject will be the responsibility of the attendant physician. However, the dose employed will depend upon a number of factors, including the age and sex of the subject, the precise disorder being treated, and its severity. Also the route of administration may vary depending upon the condition and its severity.

### EXAMPLES

The invention will be further described in the following examples, which do not limit the scope of the invention described in the claims. The following examples illustrate the characterization of the immunomodulators described herein on macrophages in vitro and in vivo in animal models of inflammation

### Example 1—Glibenclamide inhibits macrophage production of TNF and IL-12

To determine the effects of glibenclamide on proinflammatory selection, J774.1 macrophages were exposed to control (no drug) or glibenclamide ranging in concentration from 10-300 μM. TNF. Macrophage production of IL-12 was then measured. The results are shown in Table 1. Glibenclamide, a selective inhibitor of K_{ATP} channels, blocks the production of both TNF and IL-12 (Table 1).

TABLE 1
EFFECT OF GLIBENCLAMIDE ON TNF AND IL-12
PRODUCTION BY J 774.1

| Drug | | TNF (ng/ml) | IL-12 (pg/ml) |
|---|---|---|---|
| Control (no drug) | | 43.94 ± 2.48 | 669.33 ± 45.61 |
| Glibenclamide (10 | μM) | 32.91 ± 5.02 | 546.5 ± 114.5 |
| Glibenclamide (30 | μM) | — | 457.5 ± 105.5 |
| Glibenclamide (100 | μM) | 16.53 ± 4.36 | 319.5 ± 4.5 |
| Glibenclamide (300 | μM) | — | 256 ± 17 |

The effect on TNF and IL-12 macrophage production of an agent which opens K_{ATP} channels was also measured. Diazoxide, the opener of these channels, caused a substantial increase in the release of both of these cytokines (Table 2). These data point toward an important role of K_{ATP} channel activation in the regulation of macrophage function and suggest that inhibition of these channels may be an effective anti-inflammatory strategy.

TABLE 2
EFFECT OF DIAZOXIDE ON TNF AND IL-12 PRODUCTION BY
J 774.1 MACROPHAGES STIMULATED WITH LPS/IFN-γ

| Drug | | TNF (ng/ml) | IL-12 (pg/ml) |
|---|---|---|---|
| Control (no drug) | | 181.7 ± 15.85 | 669.33 ± 45.61 |
| Diazoxide (30 | μM) | 230.39 ± 43.14 | 1060.5 ± 30.5 |
| Diazoxide(100 | μM) | 186.89 ± 10.02 | 1116.50 ± 58.50 |
| Diazoxide (300 | μM) | 318.29 ± 29.58 | 6082.00 ± 80 |
| Diazoxide (500 | μM) | 360.44 ± 37.38 | — |
| Diazoxide (1000 | μM) | — | 7129 ± 175.5 |

To further examine the effect of glibenclamide on macrophage activation, the surface expression of MHC-II molecules in response to IFN-γ was measured using flow cytometry. MHC II (I-A^{d}) expression was decreased by treatment with glibenclamide. While IFN-γ exposure increased MHC II expression in peritoneal macrophages from 12.8±1 to 55.6±4.27 (mean fluorescent intensity), cotreatment of the cells with 100 μM glibenclamide decreased the expression of MHC II to 29±2.3.

Whether the inhibition of costimulatory cytokine (TNF-α and IL-12) production by glibenclamide shifted the cytokine response from a Th1 to a Th2 direction was also examined. The results demonstrated that glibenclamide decreased the production of the Th1 cytokine IFN-γ, induced by either LPS or anti-CD3 (Table 3) in mouse spleen cells. However, glibenclamide was more potent in decreasing LPS-induced, than anti-CD-3-induced, IFN-γ. In contrast, this K_{ATP} channel blocker caused concentration-dependent augmentation of the production of the Th2 cytokine IL-4 (Table 3). Taken together, these data indicate that glibenclamide polarizes the cytokine response towards a Th2 response.

TABLE 3
EFFECT OF GLIBENCLAMIDE ON IL-4 AND IFN-γ
PRODUCTION BY SPLEEN CELLS STIMULATED WITH LPS OR
AN ANTIBODY TO ANTI-CD3

| | | Anti-CD3- | Anti-CD3- |
|---|---|---|---|
| Glibenclamide | LPS-induced IFN- | induced IFN- | induced IL-4 |
| concentration (μM) | γ (pg/ml) | γ (pg/ml) | (pg/ml) |
| 0 | 79 ± 4 | 3397 ± 134 | 39 ± 14.4 |
| 1 | 29 ± 7 | 3670 ± 138 | 59 ± 15.1 |
| 3 | 17 ± 1.5 | 3459 ± 108 | 91 ± 11 |
| 10 | 14 ± 1.5 | 3688 ± 2863 | 141 ± 30 |
| 30 | 10 ± 1.5 | 2863 ± 71 | 215 ± 43 |

### Example 2—Glibenclamide suppresses inflammatory cytokine production in vivo

The effect of glibenclamide in vivo, was assessed by an endotoxemic mouse model, in which cytokine production was induced by intraperitoneal (i.p.) LPS injection and TNF-α levels were measured from the plasma of the animals. TNF-α was measured because this cytokine appears first in vivo after LPS, while IL-12 displays a delayed time-course. Under these conditions, glibenclamide significantly suppressed the plasma TNF-α level induced by LPS: the TNF-α level in vehicle-pretreated mice was 1.08±0.28 ng/ml, whereas the TNF-α level in the glibenclamide-pretreated mice was 0.43±0.09 ng/ml (n=8 in both groups; p<0.05).

### Example 3—Amilioride inhibits macrophage release of IL-12, MIP-1a, and MIP-2

Na+/H+ exchangers, also called antiporters, are transmembrane transporters involved in multiple cellular functions, including the regulation of intracellular pH, the control of cell volume, and mitogenesis (Demaurex et al., J. Exp. Biol. 196:389-404, 1994). To determine the effect of Na+/H+ antiporters on inflammatory cytokine production, stimulated macrophages were exposed to the antiporter amilioride at concentration of 0 to 300 μM, after which production of cytokines IL-12, MIP-1α and MIP-2 was measured. The results are shown in Table 4. Amiloride was found to inhibit the release of IL- 12, MIP-1α, and MIP-2 in J774.1 macrophages. These results suggest that blockade of the Na+/H+ exchangers has anti-inflammatory effects.

TABLE 4
EFFECT OF AMILORIDE ON IL-12, Mw-1α, AND
MIP-2 PRODUCTION BY S 774.1 MACROPHAGES
STIMULATED WITH LPS/IFN-γ

| Amiloride (μM) | IL-12 (ng/ml) | MW-1α (ng/ml) | MIP-2 (ng/ml) |
|---|---|---|---|
| 0 (control) | 17.19 ± 0.23 | 9.63 ± 0.34 | 15.73 ± 0.97 |
| 10 | 15.8 ± 1.8 | — | — |
| 30 | 14.82 ± 0.42 | — | — |
| 100 | 11.43 ± 1.44 | 7.59 ± 0.57 | 11.86 ± 0.2 |
| 300 | 6.42 ± 0.38 | 5.77 ± 0.41 | 8.03 ± 0.45 |

### Example 4—Inosine inhibits in vitro macrophage release of IL-12 and TNF

To determine the effect of inosine on inflammatory cytokine production, stimulated macrophages were exposed to inosine at 0 to 1000 μM, after which production of cytokines Il-12 and TNF was measured. The results are shown in Table 5. Inosine was found to inhibit the release of these cytokines. These results suggest that inosine has anti-inflammatory effects.

TABLE 5
EFFECT OF INOSINE ON IL-12 AND TNF PRODUCTION BY
PERITONEAL MACROPHAGES STIMULATED WITH LPS/IFN-γ

| Inosine (μM) | IL-12 (ng/ml) | TNF (ng/ml) |
|---|---|---|
| 0 (control) | 6.84 ± 0.39 | 16.81 ± 1.89 |
| 10 | 5.9 ± 0.19 | 10.08 ± 1.3 |
| 30 | 5.1 ± 0.14 | 8.38 ± 0.14 |
| 100 | 4.46 ± 0.62 | 7.61 ± 0.33 |
| 300 | 4.94 ± 0.06 | 5.45 ± 0.52 |
| 1000 | 4.34 ± 0.39 | 5.62 ± 0.88 |

### Example 5—Inosine inhibits inflammatory cytokine responses in vivo while increasing anti-inflammatory cytokine release

To determine whether inosine inhibits inflammatory cytokine release in vivo, male BALB/c mice were injected with inosine (100 mg/kg; i.p.) followed 30 minutes later by an i.p. injection of LPS (70 mg/kg). Plasma levels of the different cytokines were measured at various times (90 min, 2 h, 4 h, and 8 h) after the LPS challenge.

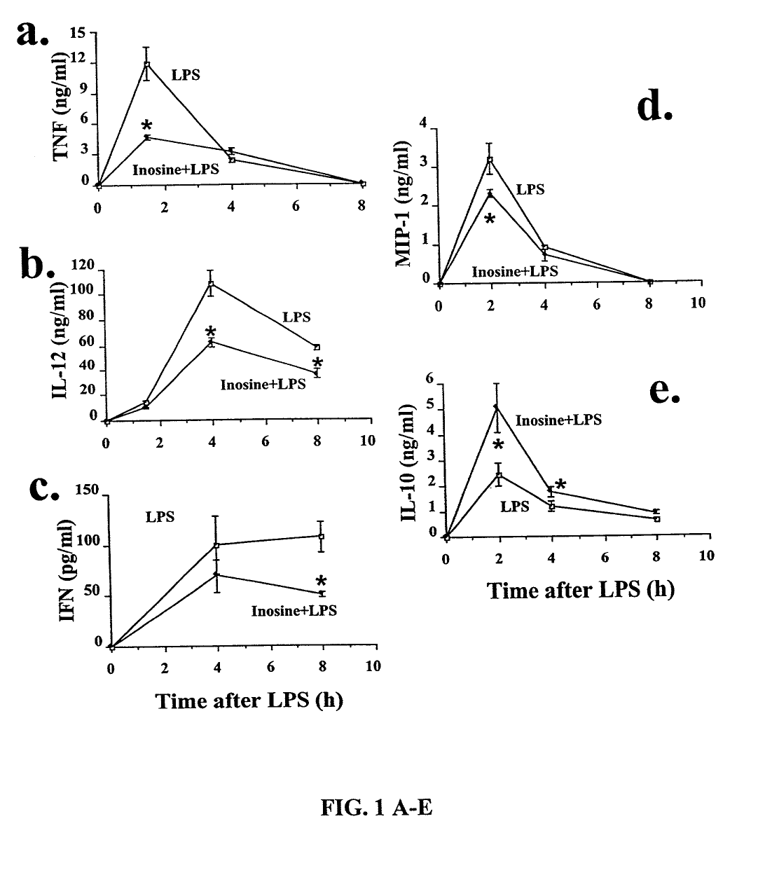

The results are shown in FIGS. 1A-E. Data are mean ±SEM of n=8 mice. The asterisk in the figure indicates p<0.05. Inosine was observed to suppress the production of TNF-α (FIG. 1A), IL-12 (FIG. 1B), IFN-γ (FIG. 1C), and MIP-1α (FIG. ID). Inosine was also shown to augment IL-10 (FIG. 1E) production in endotoxemic mice. These results were similar to the effect of inosine on cytokine release by macrophages in vitro. Notably, inosine suppressed the production of IFN-γ, which is involved in the pro-inflammatory effects of LPS. Taken together, these data demonstrated that inosine selectively and differentially altered the production of cytokines in vivo. Inosine inhibits the production of proinflammatory cytokines, but also potentiates the formation of the anti-inflammatory IL-10.

### Example 6—Inosine protects against lethal challenge of LPS in an in vivo model system

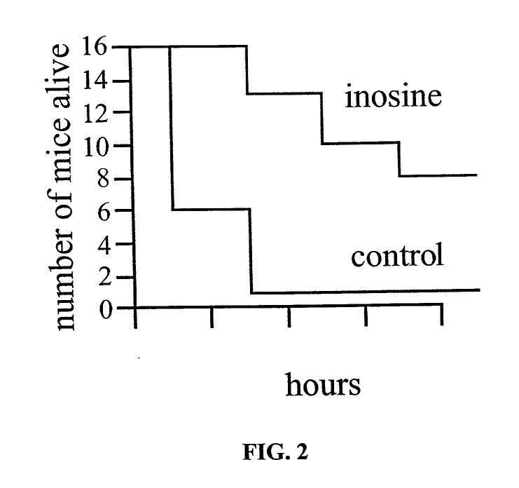

Because inosine skewed the cytokine response towards an anti-inflammatory profile, the ability of inosine to decrease LPS-induced lethality in a murine model system was investigated. BALB/c mice were pretreated with drug vehicle (physiologic saline) or 100 mg/kg inosine 30 min before the injection of 70 mg/kg of i.p. LPS. The results are shown in FIG. 2. Survival was recorded at 24, 48, 72, and 96 h after the LPS injection. Results from the summary of two different experiments are shown. N=16 animals in each group. Inosine improved survival rate at 24-96 h (p<0.05). Thus, inosine conferred significant protection in this endotoxemic model.

### Example 7—Inosine inhibits the development of diabetes-associated symptoms in an in vivo model system

Autoimmune diabetes is associated with a strong inflammatory component, along with activation of macrophages and infiltration of mononuclear cells into the pancreas. The subsequent inflammatory processes bring about the deleterious consequences of inflammation diabetes, such as islet inflammation, islet cell destruction, insulin deficiency and hyperglycemia (Rabinovitch et al., Biochem. Pharmacol. 15:1139-49, 1998; Almawi et al., J. Clin. Endocrinol. Meatab. 84:1497-502, 1999). Cytokines produced mainly by macrophages have been reported to be central mediators in the intraislet inflammatory processes.

The effect of inosine was in a rat model of streptozitocin-induced diabetes was examined. Mice were treated with streptozotocin (40 mg/kg in citrate buffer) or vehicle (citrate buffer) i.p. for 5 consecutive days to induce diabetes. Blood glucose was monitored over the following 21 days using a one-touch blood glucose meter (Lifescan). Blood glucose was measured on days 1, 7 and 21 from blood obtained from the tail vein. Hyperglycemia was defined as non-fasting blood glucose level higher than 200 mg/dL. Mice were treated simultaneously with streptozotocin injection throughout the 21 days of the experiments and with vehicle or inosine (100 mg/kg oral gavage, twice a day). Samples of pancreas were removed on day 21 and weighed before being placed into 6 mls of acid ethanol (23:7:0.45 ethanol:dH₂O:HCl) and homogenized. The pancreas samples were incubated for 72 h at 4° C. before being centrifuged. The insulin content of the supernatant was then determined using an ELISA assay.

TABLE 6 shows mean and median glucose levels, and incidence of diabetes in streptozotocin (STZ) diabetic mice receiving vehicle or inosine treatment. An “*” indicates significant reduction of circulating glucose or diabetes incidence in the inosine-treated streptozotocin rats when compared to vehicle-treated streptozotocin rats (p<0.05).

TABLE 6
CHANGE IN GLUCOSE LEVELS IN STZ DIABETIC MICE
RECEIVING VEHICLE OR INOSINE

| | Mean Blood Glucose | | | Median Blood Glucose | | | Diabetes Incidence | | |
|---|---|---|---|---|---|---|---|---|---|
| | (mg/dl) | | | (mg/dl) | | | (%) | | |
| Days | 0 | 7 | 21 | 0 | 7 | 21 | 0 | 7 | 21 |
| Control | 96 ± 4 | 103 ± 3 | 91 ± 5 | 95 | 100 | 93 | 0 | 0 | 0 |
| STZ | 93 ± 3 | 137 ± 7 | 231 ± 15 | 94 | 146 | 260 | 0 | 8 | 67 |
| (vehicle) | | | | | | | | | |
| STZ | 95 ± 4 | 112 ± 6* | 152 ± 20* | 96 | 117* | 130* | 0 | 0 | 25* |
| (inosine) | | | | | | | | | |

The relative effect of vehicle and inosine on pancreatic insulin content in STZ diabetic-mice is shown in TABLE 7. An “*” in TABLE 7 indicates significant decreases in insulin content in response to streptozotocin when compared to control, and a “#” indicates significant preservation of pancreatic insulin content in the inosine-treated streptozotocin rats (p<0.05). Pancreatic insulin content at 21 days in streptozotocin diabetic mice receiving vehicle or inosine treatment.

TABLE 7
PANCREATIC INSULIN CONTENT AT 21 DAYS IN STZ
DIABETIC MICE RECEIVING VEHICLE OR INOSINE

| | Pancreatic insulin content |
|---|---|
| | (ng insulin/mg protein) |
| Control | 68 ± 11 |
| Streptozotocin (Vehicle treated) | 6 ± 1* |
| Streptozotocin (Inosine treated) | 21 ± 4 # |

Vehicle-treated animals developed diabetes, as demonstrated by progressive hyperglycemia (Table 6) and the suppression of pancreatic insulin content (Table 7). In contrast, animals treated with inosine showed significant reductions in the incidence of diabetes, and mean and median plasma glucose levels. They also showed a significant preservation of pancreatic insulin content (Table 7). These data indicate that inosine and insosine receptor ligands are capable of suppressing the development of diabetes.

### Example 8—Inosine inhibits the development of inflammatory bowel disease symptoms in an in vivo model system

The effect of inosine in a mouse model of inflammatory disease was examined. Inosine was administered (oral administration, 100 mg/kg, 2 times a day), in a mouse model of inflammatory bowel disease induced by dextran sulfate solution (DSS). This system is well-characterized and is considered a reliable model of inflammatory bowel disease. Efficacy of a pharmaceutical compound in this model is taken as evidence that the compound is likely to be effective in human beings (Sasaki et al., Scand. J Immunol. 51:23-8, 2000; Gaudio et al., Dig. Dis. Sci. 44:1458-75, 1999; Murthy et al., Aliment Pharmacol. Ther. 13:251-60, 1999; Kimura et al., Arzneimittelforschung 48:1091-96, 1998; Dieleman et al., Scand. J Gastroenterol. Suppl. 223:99-104, 1997).

Symptoms associated with the DSS model were induced as follows. Mice were subjected to a drinking water containing 5% DSS for 10 days, in the presence (n=10) or absence (n=10) of inosine treatment (200 mg/kg/day, orally). At the end of 10 days, animals were evaluated for the incidence of bloody diarrhea, for colon shortening, and colon histopathology. Colonic myeloperoxidase and malondialdehyde levels were measured. These parameters provide a good cross-section of the functional and inflammatory changes associated with the current model of inflammatory bowel disease.

TABLE 8
EFFECT OF INOSINE ON PARAMTERS ASSOCIATED
WITH COLITIS IN DSS MICE

| Functional or | Control | IBD | IBD (DSS) |
|---|---|---|---|
| inflammatory parameter | (no DSS) | (DSS) | with inosine |
| Weight loss of the animals at | −7 ± 3 | 20 ± 2 | 13 ± 1* |
| 10 days (%) | | | |
| Colonic length | 6.0 ± 0.2 | 4.2 ± 0.1 | 5.1 ± 0.1* |
| Incidence of rectal bleeding (%) | 0 | 90 | 20* |
| Gut histological damage (1-4 scale) | 0 | 3.6 ± 0.4 | 1.2 ± 0.4* |
| Gut myeloperoxidase levels | 52 ± 9 | 296 ± 87 | 88 ± 14* |
| (mU/mg protein) | | | |
| Gut malondialdehyde levels | 1.9 ± 0.2 | 3.7 ± 0.8 | 2.1 ± 0.5* |
| (nmol/mg protein) | | | |

The results are shown in Table 8. The data demonstrate the protective effect of inosine on functional and inflammatory parameters of colitis. Significant protective effect of inosine in the presence of DSS is indicated as *p<0.05, when compared to the values with DSS alone in the absence of inosine.

Inosine treated mice responded to DSS with an improved colonic function, reduced colon shortening, and reduction in the inflammatory response in the gut.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pharmaceutical composition comprising a safe and therapeutically effective of inosine or a ligand for an inosine binding site and a pharmaceutically effective carrier.

2. The pharmaceutical composition of claim 1, wherein said pharmaceutical composition is formulated for treating inflammatory bowel disease in a subject suffering from or at risk for inflammatory bowel disease.

3. The pharmaceutical composition of claim 1, wherein said pharmaceutical composition comprises a therapeutic amount of inosine or a ligand for a inosine binding site; and a pharmacologically and bowel compatible carrier, adapted for delivery of the inosine or ligand for an insosine binding site to the bowel of said subject, said carrier being selected from the group consisting of: a foam suitable for rectal administration, a suppository base which surrounds the inosine or ligand for an insosine binding site, and an orally ingestible time-release substance which withstands degradation by the gastric acids of the stomach and releases the inosine or ligand for an inosine binding site in the bowel.

4. The pharmaceutical composition of claim 3, wherein said inosine or ligand for an inosine binding site is present in the composition in an amount ranging from 2 grams to 5 grams.

5. The pharmaceutical composition of claim 3, wherein the foam comprises inosine or a ligand for an inosine binding site, a surfactant, an adjuvant and a blowing agent.

6. The pharmaceutical composition of claim 3, wherein the carrier comprises one or more substances selected from the group consisting of propylene glycol, emulsifying wax, polyoxyethylene-10-stearyl ether, ethoxylated cetyl alcohol, ethoxylated stearyl alcohol, stearath-10, cetyl alcohol, methyl paraben, propyl paraben, trolamine, purified water, cetyl alcohol, polyoxyethylene-10-stearyl ether, propylene glycol, dry ethanolamine, de-ionized water and suitable propellents.

7. The pharmaceutical composition of claim 3, wherein the suppository base is selected from the group consisting of theobroma oil, glycerinated gelatin, hydrogenated vegetable oil, polyalkyl glycol, fatty acid ester of polyalkylene glycol, coconut oil base, hydrogenated fatty acid, hydrogenated vegetable oil, monoglyceride, cocoa butter, petroleum oil, beeswax, glycerine, polyethylene glycol 600 dilaurate, hydrogenated cocoa glyceride and polyethylene glycol.

8. The pharmaceutical composition of claim 3, wherein the time release substance is selected from the group consisting of an acrylic-based resin coating, a methacrylic acid copolymer, an acrylic-based resin mixed with a suitable non-medicinal carrier selected from the group consisting of lactose, magnesium stearate, polyethylene glycol, polyvinyl pyrolidone, or sodium starch glycolate, cellulose or ethyl cellulose, a matrix composition comprised of a hydrophilic polymer and an enteric polymer, a cellulose derivative, polyvinyl acetate phthalate, or polyvinyl acetate phthalate mixed with a plasticizer, a polysaccharide which is decomposable in the bowel, a locust bean gum or a guar gum, a film-forming polymer having hydrophilic groups, a film-forming acrylic polymer in admixture with a polysaccharide comprising from 30 to 100% by weight of at least one monomer selected from the group consisting of lower alkyl esters of acrylic acid and lower alkyl esters of methacrylic acid, a hydrocolloid gum obtained from a higher plant, and an anionic carboxylic polymer which does not dissolve at a pH below about 4 but is soluble at a pH ranging from about 4 to about 7.5.

9. The pharmaceutical composition of claim 3, wherein the foam comprises 0.5 to 5 grams of inosine as the active ingredient and 20 g of a foam containing propylene glycol, emulsifying wax, polyoxyethylene- 10-stearyl ether, cetyl alcohol, methylparaben and propylparaben, trolamine, purified water and inert propellents, dichlorodifluoromethane or dichlorotetrafluoroethane.

10. The pharmaceutical composition of claim 3, wherein the composition comprises between about 0.1% by weight to about 90% by weight of said immunomodulator coated with about 5% by weight to about 29% by weight of a hydrophilic polymer, and from about 0.5% by weight to about 25% by weight of an acrylic polymer which dissolves at a pH in the range of about 5.0 to about 7.5.

11. The pharmaceutical composition of claim 3, wherein said immunomodulator is present in a unit dosage form adaptable for oral administration

12. The pharmaceutical composition of claim 3, wherein said unit dosage form is effective to relieve a symptom of inflammatory bowel disease without dose-limiting systemic toxicity.

13. The pharmaceutical composition of claim 3, wherein said immunomodulator is enterically coated so as to be released in the terminal portion of the ileum and in the colon.

14. The pharmaceutical composition of claim 3, wherein said immunomodulator is present in a capsule or a tablet.

15. The pharmaceutical composition of claim 3, wherein the inflammatory bowel disease is ulcerative colitis.

16. An enema formulation comprising an amount of inosine or an inosine analog in an amount effective to relieve a symptom of inflammatory bowel disease without dose-limiting systemic toxicity.

17. The enema formulation of claim 16, wherein said formulation is provided in combination with a flowable carrier, which amount is released in the lower intestinal tract.

18. The enema formulation of claim 16, wherein the inflammatory bowel disease is ileitis, ulcerative colitis, and Crohn's disease.

19. The enema formulation of claim 16, wherein the amount of inosine or ligand for an inosine binding site is about 150-1000 mg.

20. The enema formulation of claim 17, wherein the flowable carrier is water.

21. The enema formulation of claim 17, wherein the flowable carrier is alcohol.

22. The enema formulation of claim 17, wherein the flowable carrier is an aqueous-alcohol fluid.

23. The enema formulation of claim 17, wherein the flowable carrier is thickened with gums, acrylates, or modified celluloses.

24. The enema formulation of claim 17, further comprising a lubricant.

25. The enema formulation claim 17, further comprising an effective amount of a foaming agent.

26. The enema formulation of claim 17, wherein said formulation is suitable for delivery from a prefilled bag or syringe.

27. The enema formulation claim 17 suitable for delivery from a pressurized container.
